(19) United States
(12) Patent Application Publication
(10) Pub. No.: US 2025/0297015 A1
(43) Pub. Date: Sep. 25, 2025

(54) DRUG CONDITIONING REGIMEN FOR SICKLE CELL DISEASE

(71) Applicant: NEW YORK BLOOD CENTER, INC., New York, NY (US)

(72) Inventor: Patricia Shi, New York, NY (US)

(21) Appl. No.: 18/864,455

(22) PCT Filed: May 10, 2023
(86) PCT No.: PCT/US2023/066829

§ 371 Date: Nov. 8, 2024

Related U.S. Application Data
(60) Provisional application No. 63/340,317, filed on May 10, 2022.

(51) Int. Cl. C07K 16/28; A61K 31/4439; A61K 31/616; A61K 48/00; A61P 7/06

(52) U.S. Cl. CPC C07K 16/2854; A61K 31/4439; A61K 31/616; A61K 48/00; A61P 7/06

(57) ABSTRACT

Disclosed herein are methods of pre-conditioning a subject with sickle cell disease in advance of plerixa for therapy comprising administration of an inhibitor of the polymerization of hemoglobin S and optionally aspirin and a P-selectin inhibitor; wherein the inhibitor of the polymerization of hemoglobin S is voxelotor, and wherein the P-selectin inhibitor is crizanlizumab or inclacumab.

### CROSS-REFERENCE TO RELATED APPLICATION

The present application claims the benefit of U.S. Provisional Patent Application 63/340,317 filed May 10, 2022, the entire contents of which are incorporated herein.

### BACKGROUND

Sickle cell disease (SCD) is a painful inherited hemoglobinopathy which causes acute and chronic vaso-occlusion in various organs and tissues in the body, as well as early mortality. Gene therapy has emerged as a potentially curative treatment for SCD. Gene modification of an adequate number of hematopoietic stem cells (HSC) and hematopoietic progenitor cells (HPC) for gene therapy requires HSPC mobilization, with or without apheresis collection. Current ex vivo gene therapy protocols require the patient to undergo conditioning for HSPC mobilization and collection via several months of chronic red cell transfusions (red cell exchange). However, red cell transfusion poses transfusion-specific risks such as delayed hemolytic transfusion reactions and hyperhemolysis. Thus, improved methods of hematopoietic stem and progenitor cells (HSPC) conditioning are needed in SCD patients.

### SUMMARY

Disclosed herein are methods for pre-conditioning a subject with sickle cell disease in advance of plerixafor therapy, the method comprising administering an inhibitor of the polymerization of hemoglobin S for one to three months prior to initiating plerixafor therapy; wherein as a result of the pre-conditioning with an inhibitor of the polymerization of hemoglobin S, mobilization of hematopoietic stem and progenitor cells (HSPC) are from the bone marrow to the peripheral blood in the subject is increased compared to mobilization without pre-conditioning with the inhibitor of the polymerization of hemoglobin S.

Also disclosed herein are methods for mobilizing hematopoietic stem and progenitor cells (HSPC) in a subject with sickle cell disease comprising: administering an inhibitor of the polymerization of hemoglobin S for one to three months prior to initiating plerixafor therapy; wherein as a result of the administration, the number of vaso-occlusive crises in the subject are reduced.

In some embodiments, the inhibitor of the polymerization of hemoglobin S is voxelotor.

In some embodiments, the method further comprises administering a P-selectin inhibitor. In some embodiments, the P-selectin inhibitor is crizanlizumab or inclacumab. In some embodiments, the crizanlizumab or inclacumab is administered for 1-4 days

In some embodiments, the method further comprises administering aspirin. In some embodiments, the aspirin is at a dose of about 81 mg/day. In some embodiments, the aspirin is administered for 1-30 days prior to plerixafor administration.

In some embodiments, the method further comprises administering both a P-selectin inhibitor and aspirin.

In some embodiments, the methods further comprise administering plerixafor after pre-conditioning with the inhibitor of the polymerization of hemoglobin S.

In some embodiments, the subject is not scheduled for red blood cell transfusion for the specific purpose of pre-conditioning for plerixafor mobilization.

In some embodiments, the subject receives gene therapy for sickle-cell disease after completion of plerixafor therapy.

### BRIEF DESCRIPTION OF THE DRAWINGS

FIG. 1A-D depicts the murine peripheral blood response to GBT-1118, the murine analog of voxelotor, and control, in individual mice before treatment, 2 weeks after treatment, and 3 weeks after treatment in hemoglobin (Hb; FIG. 1A), hematocrit (HCT; FIG. 1B), reticulocytes (Retic %; FIG. 1C), and white blood cells (WBC; FIG. 1D). Each data point represents a single mouse.

FIG. 2 depicts the murine peripheral blood WBC response to plerixafor (P)+GBT-1118 (and control+plerixafor) with regard to total WBC (WBC), neutrophils, and monocytes. Each data point represents a single mouse.

FIG. 3A-D depicts the effects of GBT-1118, with and without plerixafor (and GBT-1118 controls), on murine peripheral blood hematopoietic progenitor cells (HPC). FIG. 3A depicts LSKF cells (Lin Sca-1⁺c-kit⁺Flt3⁻CD135⁻) as a percentage of CD45⁺ cells. FIG. 3B depicts the concentration of LSKF cells in peripheral blood. FIGS. 3C and 3D depict LSKF cells as a percentage of Lin-cells, with and without CD45+ gating. Each data point represents a single mouse.

FIG. 4A-F depicts the effects of GBT-1118, with and without plerixafor (and GBT-1118 controls), on murine bone marrow HPCs and HSCs. FIG. 4A depicts the bone marrow HSC as a percentage of Lin cells, FIG. 4B depicts the number of bone marrow HSCs per fermur. FIG. 4C depicts the bone marrow HSCs as a percentage of live cells. FIG. 4D depicts the bone marrow LSK cells (Lin⁻Sca-1⁺c-kit⁺) as a percentage of Lin cells. FIG. 4E depicts the number of bone marrow LSK cells per femur. FIG. 4F depicts the bone marrow LSK cells as a percentage of live cells. Each data point represents a single mouse.

FIGS. 5A and 5B depict the effects of plerixafor (P)+GBT-1118 (and GBT-1118 control) on aged neutrophils in murine peripheral blood. FIG. 5A depicts the percentage of aged neutrophils of total neutrophils. FIG. 5B depicts the concentration of aged neutrophils in peripheral blood. Each data point represents a single mouse.

FIG. 6A-C depicts the effects of GBT-1118, with and without plerixafor (and GBT-1118 controls), on expression of plasma inflammatory markers in murine peripheral blood. FIG. 6A depicts concentration of plasma sVCAM-1. FIG. 6B depicts concentration of plasma P-selectin. FIG. 6C depicts concentration of plasma E-selectin. Each data point represents a single mouse.

### DETAILED DESCRIPTION

Bone marrow harvesting was the initial approach for hematopoietic stem and progenitor cell (HSPC) collection in sickle-cell disease (SCDO, with evidence supporting its utility in both animal models and in vitro studies utilizing patients' material. However, bone marrow harvesting results in suboptimal yields of high purity hematopoietic stem cells (HSC) at the end of collection and processing, along with substantial pain after each harvest, and most subjects required two or three harvests to yield sufficient cell doses for manufacturing.

The success of gene therapy in SCD relies on several key factors; these include the source, quality and number of transduced cells, the choice of the conditioning regimen, the level of therapeutic transgene expression, and the quality of the bone marrow (BM) microenvironment at the time of harvest and transplantation. Approximately 2 to 3×10⁶ CD34⁺ HSPC/kg are required for a successful outcome in autologous hematopoietic stem cell transplantation (HSCT). For gene therapy, considering cell processing and manufacturing losses, ˜6×10⁶ CD34⁺ cells/kg is typically required for the collected product. The recovery of HSPC from SCD patients' bone marrow (BM) is peculiarly low, requiring multiple BM harvests (each requiring an exchange transfusion program before general anesthesia) to obtain enough cells to transduce and transplant.

Filgrastim- (or granulocyte colony-stimulating factor) based mobilization and apheresis is the standard method for HSPC collection in healthy adult donors, yet this approach in SCD is associated with high rates of adverse events requiring hospitalization, including vaso-occlusive crises, multi-organ failure, and even death, prompting calls for a moratorium on its use for HSPC mobilization in SCD.

As an alternative to filgrastim, plerixafor can effectively mobilize HSPC. Plerixafor directly inhibits the binding of stroma-cell-derived factor-1a to its CXCR4 chemokine receptor on HSPC, releasing HSPC from the BM niche.

However, all of these cell collection protocols require prior red blood cell transfusion or exchange to achieve <30% sickle hemoglobin (HbS) prior to HSPC mobilization.

One problem associated with current protocols is inadequate HSPC mobilization with transfusion conditioning. Seventy-three percent of patients on red cell transfusion conditioning still require two to five days of apheresis, and 47% of patients still need at least two mobilization cycles. The disclosed conditioning regimen will improve the efficacy of HSPC mobilization compared to transfusion.

Another problem associated with current protocols is persistent vaso-occlusive events with transfusion conditioning. Twenty percent of transfused patients still have severe vaso-occlusive pain following HPC mobilization and collection. The disclosed conditioning regimen will increase the safety of HSPC mobilization and collection with regard to vaso-occlusive patient adverse events.

Another problem associated with current protocols are transfusion-specific patient adverse events. Red cell transfusion poses alloimmune risks such as delayed hemolytic transfusion reactions and hyperhemolysis. The disclosed conditioning regimen does not involve transfusion and thus has none of these alloimmune risks.

Another problem associated with current protocols is transfusion availability. Sickle cell disease is most prevalent in sub-Saharan Africa but in these countries, transfusions are not easily available or accessible. The disclosed conditioning regimen involves the patient taking oral tablets for the majority of the period of conditioning.

Yet another problem associated with current protocols is transfusion-related transmissible disease. In Sub-Saharan Africa, the safety of chronic red cell transfusion is challenged by the risks of infection, specifically transfusion-transmitted infections. The disclosed conditioning regimen does not involve transfusion and thus has no risk of transfusion-transmitted infection.

Disclosed herein is pre-conditioning with an inhibitor of the polymerization of hemoglobin S as an alternative to red cell transfusion to improve the safety and efficacy of HSPC mobilization and collection. In some embodiments, the inhibitor of polymerization of hemoglobin S is voxelotor. In some embodiments, the pre-conditioning comprises voxelotor and one or both of low-dose aspirin and a P-selectin inhibitor prior to plerixafor. Similar to red cell transfusions, voxelotor increases hemoglobin concentrations and may thus improve the bone marrow microenvironment. Incorporation of a P-selectin inhibitor and low dose aspirin has an HSPC mobilizing effect as well as offers protection from vaso-occlusive complications. The disclosed pre-conditioning with voxelotor prior to plerixafor offers a more efficient and safer method than transfusion for HPC mobilization prior to gene therapy in SCD.

In some embodiments, the dose of voxelotor is about 1,000-2,500 mg/day. In some embodiments, the dose of voxelotor is 1,000-2,000 mg/day, 1,100-1,900 mg/day, 1,200 -1,800mg/day, 1,300-1,700 mg/day, or 1,400-1,600 mg/day, or a range defined by any two of the foregoing values. In some embodiments, the dose of voxelotor is about 1000 mg/day, about 1,200 mg/day, about 1,400 mg/day, about 1,500 mg/day, about 1,600 mg/day, about 1,800 mg/day, about 2,000 mg/day, about 2,200 mg/day, about 2,400 mg/day, or about 2,500/day. In some embodiments, the dose of voxelotor is less than 1,000 mg/day. In some embodiments, the dose of voxelotor is about 1,500 mg/day. In some embodiments, the dose of voxelotor is greater than 2,500 mg/day. Voxelotor is administered daily for about 1-3 months prior to initiation of plerixafor administration. In some embodiments, voxelotor is administered daily for about 1 month prior to the initiation of plerixafor administration. In some embodiments, voxelotor is administered daily for about 2 months prior to the initiation of plerixafor administration. In some embodiments, voxelotor is administered daily for about 3 months prior to the initiation of plerixafor administration.

In some embodiments, the disclosed methods may include the administration of aspirin (acetysalicylic acid). In some embodiments, the dose of aspirin is about 81 mg. In some embodiments, aspirin is administered daily for at least 10 days prior to the initiation of plerixafor administration. In some embodiments, aspirin is administered daily for at least 14 days prior to the initiation of plerixafor administration. In some embodiments, aspirin is administered daily for at least 20 days prior to the initiation of plerixafor administration. In some embodiments, aspirin is administered daily for at least 30 days prior to the initiation of plerixafor administration.

In some embodiments, the disclosed methods may include the administration of a P-selectin inhibitor. In some embodiments, the P-selectin inhibitor is an antibody which blocks the interaction between P-selectin and P-selectin glycoprotein ligand-1. In some embodiments, the P-selectin inhibitor is crizanlizumab or inclacumab. In some embodiments, crizanlizumab or inclacumab is administered from about 1-7 days prior to the initiation of plerixafor administration. In some embodiments, crizanlizumab or inclacumab is administered as a single dose about 12-48 hours prior to the initiation of plerixafor administration. In some embodiments, crizanlizumab or inclacumab is administered as a single dose about 48-120 hours prior to the initiation of plerixafor administration.

In some embodiments, plerixafor is administered at a dose of about 80-480 μg/kg daily for up to four consecutive days. In some embodiments, the dose of plerixafor is about 100-400 μg/kg, about 200-400 μg/kg, about 300-400 μg/kg. In some embodiments, the dose of plerixafor is about 250-350 μg/kg, or a range defined by any two of the foregoing values.

As a result of the pre-conditioning with the inhibitor of the polymerization of hemoglobin S, mobilization of HSPC are from the bone marrow to the peripheral blood in the subject is increased compared to mobilization without pre-conditioning with the inhibitor of the polymerization of hemoglobin S, or with pre-conditioning by other means. In some embodiments, the mobilization is increased by 10%, by 20%, by 30%, by 40%, by 50%, by 60%, by 70%, by 80%, by 90%, by 100%, or by more than 100% compared to mobilization without pre-conditioning with the inhibitor of the polymerization of hemoglobin S or with pre-conditioning by other means.

Thus, disclosed herein are methods of increasing HSPC mobilization and recovery in subjects with sickle cell disease to (1) increase the degree of HSPC mobilization; (2) increase the safety of HSPC mobilization and collection in regard to vaso-occlusive patient adverse events; (3) decrease infectious disease risks; (4) remove alloimmunization risk; and (5) improve accessibility of gene therapy, all in comparison to the existing red blood cell transfusion regimen.

### EXAMPLES

### Example 1

### Utility of Voxelotor as Conditioning for Hematopoietic Progenitor Cell Mobilization in Sickle Cell Disease

This study uses a SCD mouse model, SS Townes mice, to evaluate the effects of GBT-1118, the murine analog of voxelotor, to mobilize hematopoietic progenitor cells (HPC). GBT-1118 (approximately 16 mg/mouse/day) was administered via chow (4 grams of GBT-1118 per kg of chow) daily for a month. The mice were then administered a single dose of plerixafor (10 mg/kg) via subcutaneous injection. The GBT-1118+plerixafor group (n=6) were compared to the following control groups: control chow+plerixafor (n=5), GBT-1118 alone (n=3), and control chow alone (n=3). Peripheral blood was collected in all groups prior to and two-and three-weeks post treatment with GBT-1118 (or control) to assess hemoglobin (Hgb), hematocrit (Hct), aged neutrophils, and circulating HPCs. One to two hrs after plerixafor, peripheral blood was collected for circulating HPC (LSKF) enumeration. Assay results in the groups are compared using ANOVA or Kruskal-Wallis testing.

Also evaluated were bone marrow HPCs and hematopoietic stem cells (HSCs) with and without plerixafor and peripheral blood inflammatory markers (aged neutrophils, sVAM-1, P-selectin, and E-selectin) with and without plerixafor.

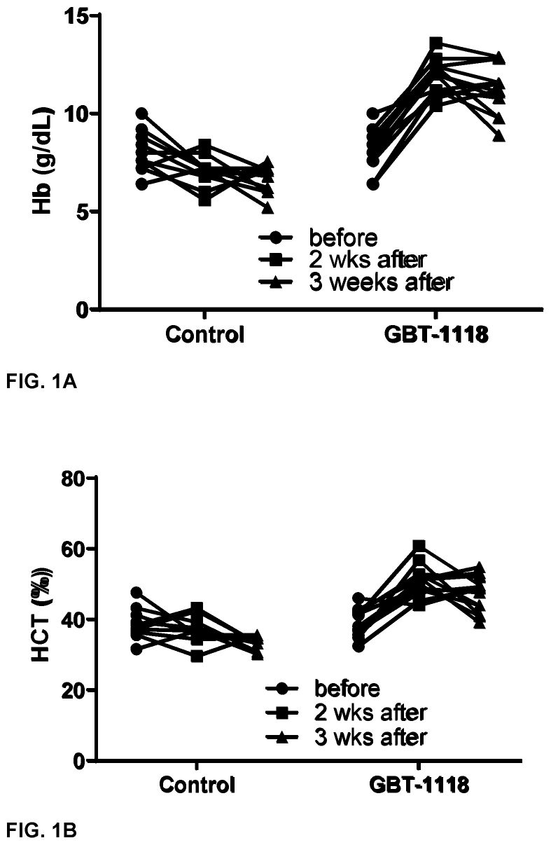

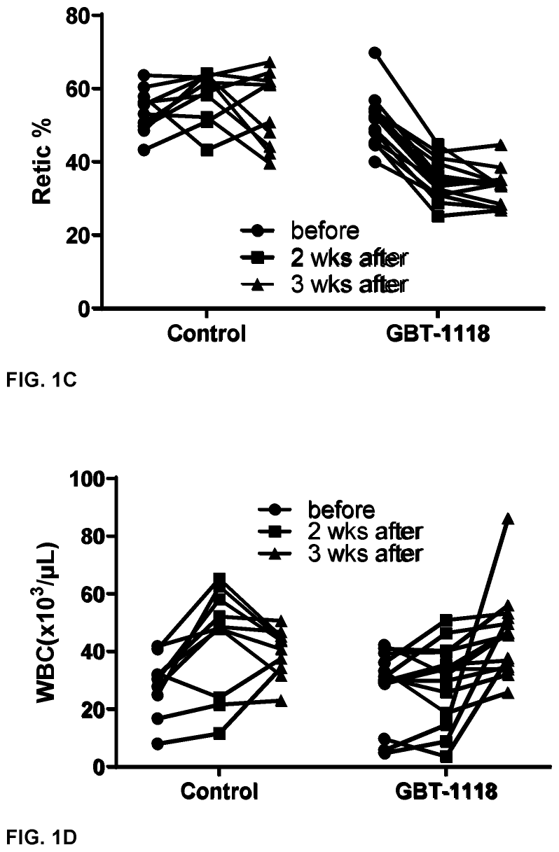

As expected, there was an increase in Hb/Hct (FIGS. 1A and 1B) and a decrease in the percentage of reticulocytes (retic %) (FIG. 1C) after GBT-1118 treatment. The number of white blood cells (WBC) was essentially unchanged (FIG. 1D).

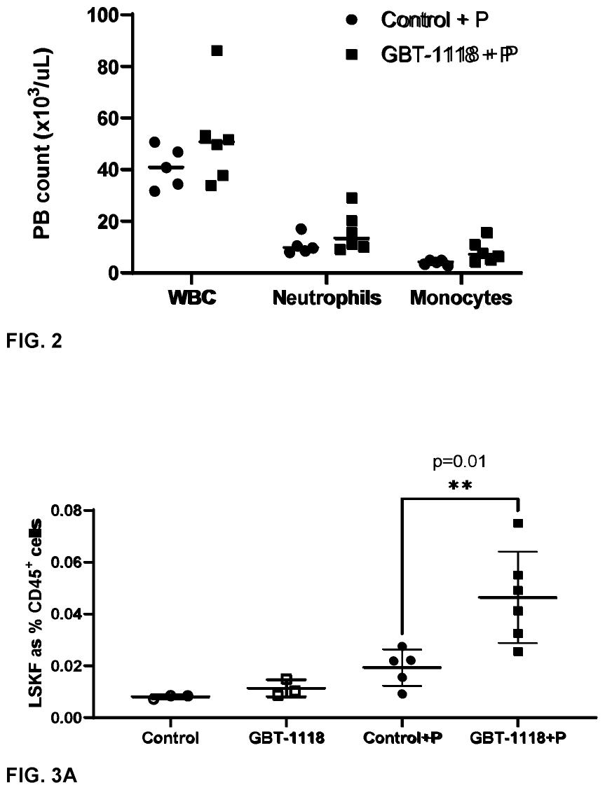

There was no significant differences in the concentration of peripheral blood WBC (total WBC, neutrophils, and monocytes) between animals receiving control+plerixafor alone versus GBT-1118+plerixafor (FIG. 2).

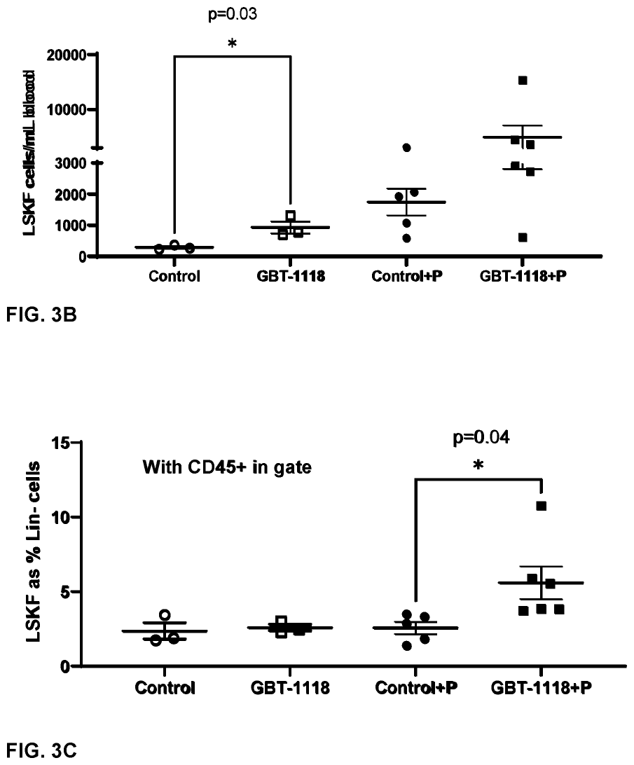

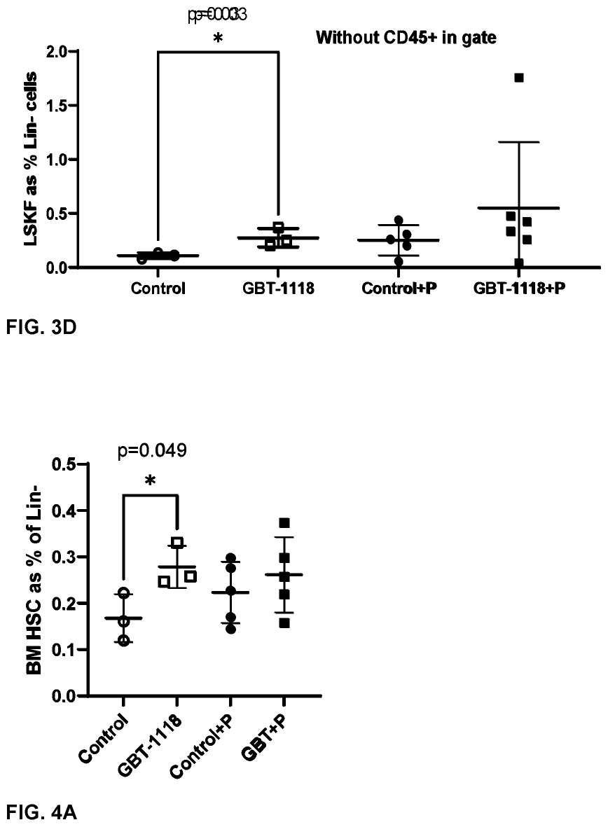

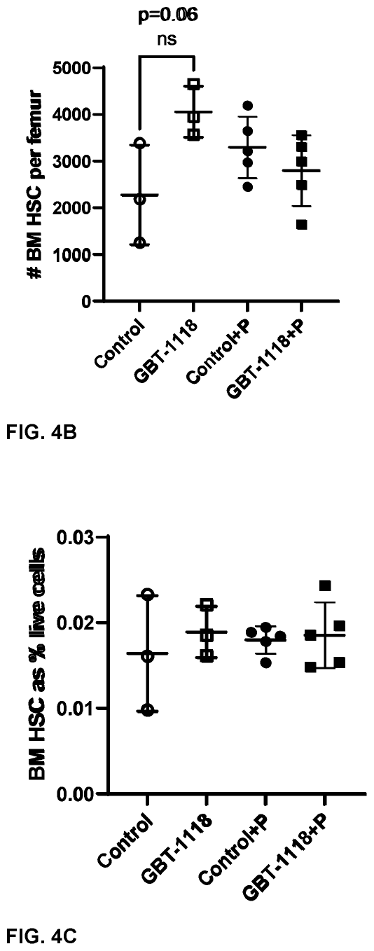

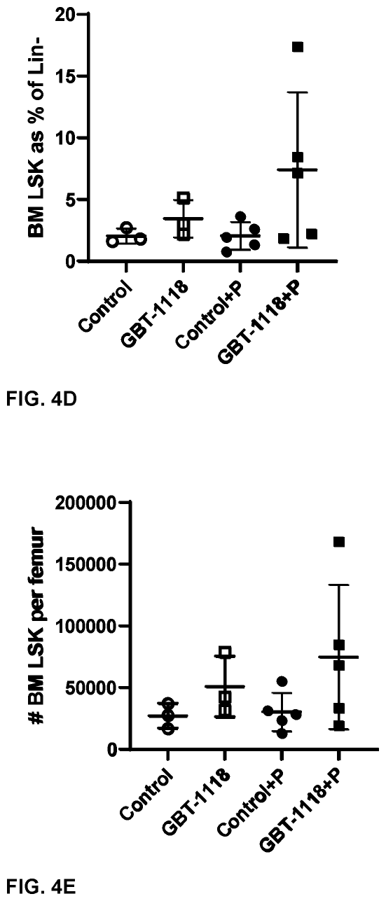

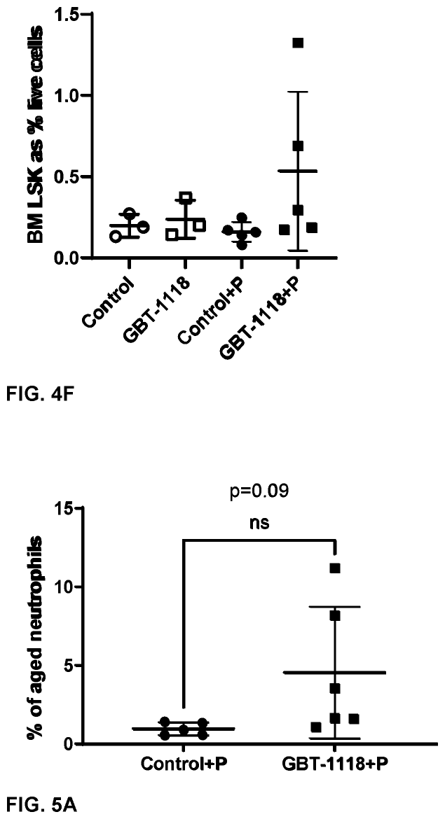

Gating with and without CD45⁺ was performed due to the erythroid hyperplasia seen in SCD, where CD45⁺ expression decreases along with erythroid maturation (Boulais et al. Immunity 49:627-639, 2018) and the degree of erythroid hyperplasia in each mouse was unknown. The percentage of CD45⁺ cells ranged from 60-80% per sample. When CD45⁺ mature erythroid cells were gated out, the GBT-1118-treated animals, compared to the control animals, mobilized a higher percentage of the peripheral blood LSKF (Lin⁻Sca1⁺ckit⁺Flt3⁻CD135⁻) cells (FIGS. 3A and 3C). The peripheral blood LSKF cell concentration was not significantly increased in the GBT-1118+P treated mice (FIG. 3B), perhaps due to varying degrees of absolute mobilization in individual mice, as in humans. The peripheral blood LSKF cell concentration was significantly increased with GBT-1118 alone compared to control, however (FIG. 3B). As red cell transfusion tends to decrease circulating hematopoietic stem cells (HSCs) (Tang A et al, Blood 2021), this may be due to the increased bone marrow HSCs (FIGS. 4A and 4B) spilling into peripheral blood due to residual mesenchymal stem cell dysfunction.

In the bone marrow, the percentage of HSCs, as % of Lin⁻ cells, was significantly increased by treatment with GBT-1118 alone (FIG. 4A). This is consistent with the finding of increased LSKF in peripheral blood with GBT-1118 alone (FIGS. 3B and 3D) and with plerixafor mobilization (FIGS. 3A and 3C). There was a trend towards an increase in the total number of bone marrow HSC per femur with GBT-1118 versus control (FIG. 4B).

There were no significant differences in bone marrow LSK between the four groups (FIG. 4D-F). Bone marrow LSK (Lin⁻Sca1⁺ckit⁺) is a less HSC-specific measure than bone marrow HSC (Lin⁻Sca1⁺ckit+CD48⁻CD150⁺); the markers Flt3 and CD135 are not present in bone marrow as they are in peripheral blood.

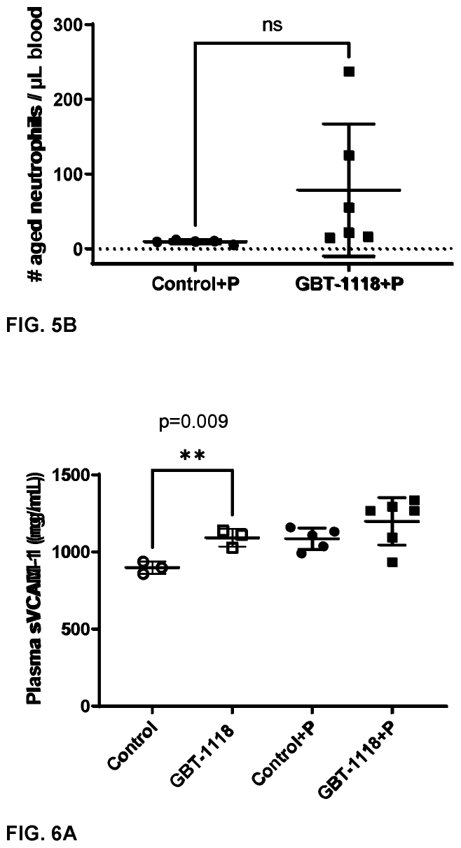

Aged neutrophils represent an overly active subset of neutrophils exhibiting enhanced αMβ2 integrin activation and neutrophil extracellular trap formation under inflammatory conditions. There is an increase in the aged neutrophil population in SCD. The effects of plerixafor with and without GBT-1118 on peripheral blood aged neutrophils (CXCR4^{high} CD62L^{low}) is depicted in FIG. 5A-B. Although there is a trend to increased aged neutrophils in GBT-1118-treated mice post-mobilization, the differences were not statistically significant, which suggests that use of voxelotor as pre-conditioning for HPC mobilization is safe.

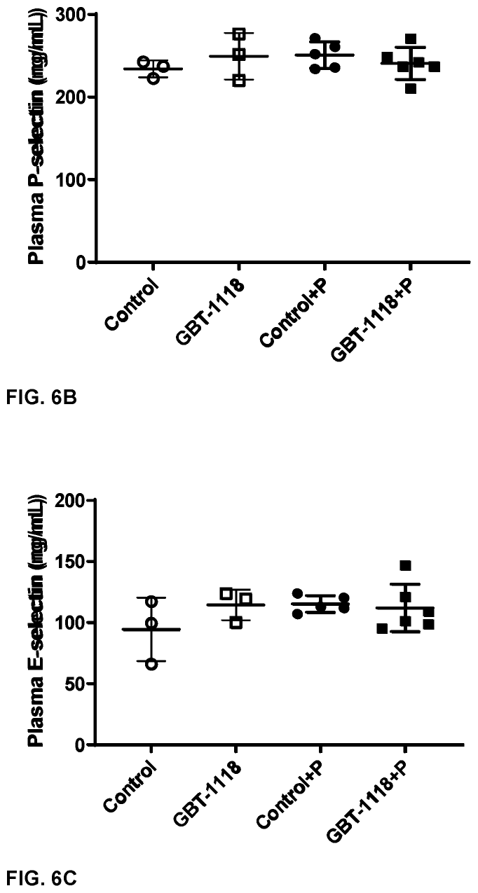

There is evidence that SCD is associated with a chronic inflammatory state. Therefore, inflammatory markers (sVCAM-1, P-selectin, and E-selectin) in the peripheral blood of mice treated with GBT-1118 alone or with plerixafor were evaluated. There was a small but significant increase in sVCAM-1 after GBT-1118 treatment (FIG. 6A). This increase may be related to increased WBC and absolute neutrophil counts (ANC). Plasma P-selectin and E-selectin were not increased with GBT-1118 alone or with plerixafor (FIGS. 6B and 6C).

Sickle mice treated with GBT-1118 alone showed increased bone marrow hematopoietic stem cells (HSCs). There was a trend towards increased total HSCs per femur also. This result suggests that voxelotor may improve bone marrow health, like that seen with blood transfusion (Tang A et al, Blood 2021). This result has implications for both chronic treatment of SCD and the auto-and allo-transplant setting in SCD.

Sickle mice treated with GBT-1118+plerixafor showed increased peripheral blood HSCs. This result suggests that voxelotor may improve HSC mobilization, which may be related to increased bone marrow HSCs due to improved bone marrow health.

Furthermore, GBT-1118+plerixafor appears safe by inflammatory marker analysis.

### Example 2

### Inclusion of Anti-Inflammatory Agents with GBT-1118 and Plerixafor

Using the methods disclosed in Example 1, GBT-1118 is administered in combination with low-dose aspirin and/or a P-selectin inhibitor.

The following groups are used for this experiment: (1) plerixafor alone; (2) GBT-1118 alone; (3) GBT-1118 pre-treatment followed by plerixafor; (4) aspirin pre-treatment followed by plerixafor; (5) P-selectin inhibitor pre-treatment followed by plerixafor; (6) GBT-1118+aspirin pre-treatment followed by plerixafor; (7) GBT-1118+P-selectin inhibitor pre-treatment followed by plerixafor; (8) GBT-1118+aspirin+P-selectin inhibitor followed by plerixafor. There will also be the appropriate control groups for each of these treatment groups.

The pre-treatment or control regimen is administered over the course of one month (daily for GBT-1118 and aspirin, and once, twice, or three times over the course of the pre-treatment for the P-selectin inhibitor). Peripheral blood will be collected in all groups prior to, and two-and three-weeks post pre-treatment to assess hemoglobin (Hgb), hematocrit (Hct), aged neutrophils, and circulating HPCs. A subset of treatment and control mice are sacrificed without being given plerixafor to examine bone marrow HPC and HSC. A subset of treatment and control mice are administered a single dose of plerixafor (10 mg/kg) via subcutaneous injection. One to two hrs after plerixafor, peripheral blood and bone marrow will be collected for HPC and HSC enumeration. Assay results in the groups are compared using ANOVA or Kruskal-Wallis testing.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A method for pre-conditioning a subject with sickle cell disease in advance of plerixafor therapy, the method comprising:
administering an inhibitor of the polymerization of hemoglobin S for one to three months prior to initiating plerixafor therapy;
wherein as a result of the pre-conditioning with an inhibitor of the polymerization of hemoglobin S, mobilization of hematopoietic stem and progenitor cells (HSPC) from the bone marrow to the peripheral blood in the subject is increased compared to mobilization without pre-conditioning with the inhibitor of the polymerization of hemoglobin S.

2. The method of claim 1, wherein the inhibitor of the polymerization of hemoglobin S is voxelotor.

3. The method of claim 1, further comprising administering a P-selectin inhibitor.

4. The method of claim 3, wherein the P-selectin inhibitor is crizanlizumab or inclacumab.

5. The method of claim 4, wherein the crizanlizumab or inclacumab is administered for 1-4 days

6. The method of claim 1, further comprising administering aspirin.

7. The method of claim 6, wherein the aspirin is at a dose of about 81 mg/day.

8. The method of claim 6, wherein the aspirin is administered for 1-30 days prior to plerixafor administration.

9. The method of claim 1, further comprises administering both a P-selectin inhibitor and aspirin.

10. The method of claim 1, further comprising administering plerixafor after pre-conditioning with the inhibitor of the polymerization of hemoglobin S.

11. The method of claim 1, wherein the subject is not scheduled for red blood cell transfusion for the specific purpose of pre-conditioning for plerixafor mobilization.

12. The method of claim 1, wherein the subject receives gene therapy for sickle-cell disease after completion of plerixafor therapy.

13. A method of mobilizing hematopoietic stem and progenitor cells (HSPC) in a subject with sickle cell disease comprising:
administering an inhibitor of the polymerization of hemoglobin S for one to three months prior to initiating plerixafor therapy;
wherein as a result of the administration, the number of vaso-occlusive crises in the subject are reduced.

14. The method of claim 13, wherein the inhibitor of the polymerization of hemoglobin S is voxelotor.

15. The method of claim 13, further comprising administering a P-selectin inhibitor.

16. The method of claim 15, wherein the P-selectin inhibitor is crizanlizumab or inclacumab.

17. The method of claim 16, wherein the crizanlizumab or inclacumab is administered for 1-4 days

18. The method of claim 13, further comprising administering aspirin.

19. The method of claim 18, wherein the aspirin is at a dose of about 81 mg/day.

20. The method of claim 18, wherein the aspirin is administered for 1-30 days prior to plerixafor administration.

21. The method of claim 13, further comprises administering both a P-selectin inhibitor and aspirin.

22. The method of claim 13, further comprising administering plerixafor after pre-conditioning with the inhibitor of the polymerization of hemoglobin S.

23. The method of claim 13, wherein the subject is not scheduled for red blood cell transfusion for the specific purpose of pre-conditioning for plerixafor mobilization.

24. The method of claim 13, wherein the subject receives gene therapy for sickle-cell disease after completion of plerixafor therapy.
